(19) United States
(12) Patent Application Publication
(10) Pub. No.: US 2015/0057232 A1
(43) Pub. Date: Feb. 26, 2015

(54) Anti-Inflammatory Composition for Modulating the Cell Response of Neutrophils and Eosinophils

(71) Applicant: Ovogenics SA, Waterloo (BE)

(72) Inventors: Pascal Melsens, Waterloo (BE); Jean Louis Charles Sainte Laudy, Saint-Junien (FR)

(21) Appl. No.: 14/377,646

(22) PCT Filed: Feb. 8, 2013
(86) PCT No.: PCT/EP2013/052536

§ 371 Date: Aug. 8, 2014

(30) Foreign Application Priority Data
Feb. 10, 2012 (BE) 2012/008

(51) Int. Cl. A61K 38/57; A23L 1/302; A23L 1/303; A23L 1/304

(52) U.S. Cl. CPC A61K 38/57; A23L 1/304; A23L 1/302; A23L 1/303
USPC 514/20.3

(57) ABSTRACT

The present invention relates to an anti-inflammatory composition intended for humans and animals for modulating the cell response of the neutrophils and of eosinophils.

The present invention relates to an anti-inflammatory composition intended for humans and animals comprising extract from eggs of quails Corturnix corturnix japonica of the B-Mina strain comprising an ovomucoid and an ovo-inhibitor of quail egg.

Ovomucoid is an enzyme inhibiting proteases (anti-protease enzyme) which is present in the egg white of birds and which includes three disulfide bridges and glucose, mannose, deoxy-glucose and sialic acid residues. This polypeptide comprises three very similar functional domains each capable of binding a protease, for example trypsin (this is then referred to as anti-trypsic activity).

The ovo-inhibitor is a glycoprotein, the structure of which is different from that of the ovomucoid. Like the ovomucoid, the ovo-inhibitor is part of the inhibitors of serine proteases. This is a powerful inhibitor of bovine trypsine, of α-chymotrypsin, of chicken chymotrypsin, of elastase, of subtilisin (from Bacillus subtilis) and of bacterial and fungal proteases.

Proteases are enzymes provided with proteolytic pro-inflammatory properties contained in the intracytoplasmic granulations of immune cells (mastocytes, basophils, neutrophils, and eosinophils for example).

On the one hand, certain proteases, among which are serine-proteases, are involved in humans in different basic physiological processes such as coagulation, activation of the complement, tissue repair and immunity. Serine-proteases also intervene in the cascade for activating basophils and mastocytes. For example, serine-proteases are present in lung mastocytes in the form of tryptase and chymase which are involved in the cascade for activating basophils and mastocytes. It was shown that anti-proteases may inhibit activation of mastocytes, like diisopropylfluorophosphate (DFP) which inhibits the release of histamine by lung mastocytes. Moreover, anti-proteases belonging to the group of serpins (antithrombin III and inhibitory C1s esterase) allow inhibition of the aggregation induced by fMLP between neutrophils and platelets. Also, the proteinase 3 (PR3) which is the target antigen of c-ANCA (cytoplasmic antineutrophil cytoplasmic antibodies) is inhibited by alpha-1 antitrypsin.

On the other hand, the proteases released by pro-inflammatory cells, for example during an inflammation, may lead to a worsening of the symptoms, such as for example the case for asthma or cystic fibrosis. It was also shown that proteases released by pro-inflammatory cells such as neutrophils may induce the production of superoxide ions and of pro-inflammatory cytokines such as IL8 leading to the recruitment of neutrophils at the site of the inflammatory reaction and thus to worsening of the symptoms, like this is the case for asthma and cystic fibrosis. In such cases, a perfect proteases/anti-proteases balance is sought in order to attenuate the symptoms and consequently it is sometimes useful to promote this balance by providing protease inhibitors, for example via an ovomucoid.

The main blood cells having a fast pro-inflammatory action (of less than one hour) are the basophils, eosinophils and neutrophils.

Basophils are poorly represented cells in humans and capable of infiltrating the tissue site of the inflammatory reaction. Basophils are powerful pro-inflammatory cells containing, in their granules, many preformed mediators such as histamine and proteases which will be released after activation by basophilic cells. The activation of basophilic cells may be triggered by mechanisms which are either non-dependent on antibodies or dependent on antibodies, and is mainly mediated by IgEs, IgGs and IgAs. This activation induces the release of mediators, in particular histamine which causes an immune response. These different mechanisms involving basophilic cells are generally observed during allergic inflammatory reactions.

Eosinophils are blood cells found in tissues at the sites of inflammatory reactions and play a major regulatory role on the immune system. Eosinophilic cells have membrane receptors for IgGs, for IgAs and for IgEs as well as for various cytokines (IL2, IL3, IL5, IFNγ). Activation of eosinophils results in their degranulation with release of many mediators among which are proteins (MBP, ECP and EDN) and enzymes (protease, peroxidase, collagenases, β-glucuronidase, lysophosphatase, phospholipases, histaminases, catalases, esterases, hexosaminidases, . . . ). Activation of eosinophils further leads to the secretion of oxygenated radicals involved in oxidative stress and to the release of derivatives of the cyclo-oxygenase route. Eosinophilic cells are involved in many pathologies such as gastritis, esophagitis, eosinophilic proctocolite, ulcerative colitis, Crohn's disease, Churg-Strauss syndrome, eosinophilic pneumonia and diseases from parasites. However, the most frequent causes of eosinophilia are allergic inflammatory diseases such as atopical dermatitis, allergic rhinitis and asthma.

Neutrophils are essential pro-inflammatory cells which are the first recruited cells after tissue damages and in response to infectious agents or to allergens. These cells are also involved in oxidative stress and are thus capable of releasing extremely active oxygenated derivatives and toxic for tissues on the site of the inflammatory reaction. Neutrophils may be activated via the IgA, IgG and even IgE route.

Neutrophilic and eosinophilic cells are capable of releasing, after their activation, proteolytic enzymes (proteases) with sometimes deleterious effects on the tissues, which was notably demonstrated in the case of asthma. This is why a proteases/anti-proteases balance is sought for attenuating the symptoms, which may be obtained by providing inhibitors of proteases, for example via an ovomucoid inhibiting proteases.

The use of the quail egg ovomucoid as an inhibitor of proteases in inflammatory reactions is known from the state of the art. Prior studies have demonstrated the inhibiting effect of quail egg ovomucoid on the activation of human basophils stimulated by a bacterial peptide, N-formyl-methionyl-leucyl-phenylalanine (fMLP).

For example, document FR2901137 describes several medicinal galenic forms intended for human and animal medicine, formed by one or several natural inhibitors of protease enzymes. This document thus discloses medicinal compositions based on natural protease inhibitors extracted from quail eggs, associated with vitamins and/or minerals and/or with amino acids and incorporated in an excipient. These medicinal compositions have a global anti-inflammatory activity targeted on all tissues and organs, a non-specific anti-inflammatory activity based on the inhibition of trypsin or of its homologs (elastase, tryptase, . . . ) and a specific anti-allergic anti-inflammatory activity based on the inhibition of the release of chemical mediators by mastocytes or basophils. These medicinal compositions may appear in the form of tablets comprising one or several natural inhibitors of serine-proteases, like the ovomucoid and the ovo-inhibitor extracted from the quail egg white of species Corturnix corturnix japonica of strain B-Mina and various constituents such as for example vitamins and/or amino acids. More particularly, these medicinal compositions are indicated for preventing and treating inflammations.

Document FR2813531 also deals with medicinal compositions comprising a quail egg homogenate associated with trace elements and having an anti-protease and anti-inflammatory activity. These medicinal compositions have a non-specific anti-allergic activity based on the anti-protease activity of quail egg ovomucoid, an anti-inflammatory activity by inhibition of the release of histamines by mastocytes and basophils and a dermatological activity by action on the proteases of specialized cells of the dermis, for example neutrophils. These medicinal compositions may appear as tablets consisting of a homogenate of quail eggs (yolk and white) having an anti-protease, anti-inflammatory and anti-infectious activity related to the ovomucoid fraction contained in the white of the egg and one or several incorporated elements. These medicinal compositions are notably indicated for preventing and treating allergic diseases, respiratory infections and certain skin affections.

Document FR2939044 describes medicinal compositions consisting of a donkey milk lyophilisate mixed with one or several natural inhibitors of human protease enzymes such as the ovomucoid and ovo-inhibitor extracted from quail eggs from the species Corturnix corturnix japonica of strain B-Mina. This formulation is indicated in preventing and treating pathologies affecting the immune system but also for applying food supplements or cosmetic products. Indeed, this formulation has a strong nutritional value, a global anti-inflammatory activity on all tissues and organs, a non-specific anti-inflammatory activity related to the inhibitors of human proteases, a specific anti-inflammatory and anti-allergic activity by the inhibition of the release of chemical mediators excreted by basophilic cells and mastocytes and a dermatological anti-inflammatory activity by exerting an inhibitory action on the enzymes of the inflammation.

As regards the prior art, there exists on the one hand an anti-protease activity of ovomucoids and of ovo-inhibitors of eggs of the quail Corturnix corturnix japonica of strain B-Mina, acting on human proteases involved in inflammatory and allergic mechanisms such as for example on trypsin and its homologs (elastase, tryptase, . . . ). This anti-protease activity therefore acts on proteases after their release by pro-inflammatory cells such as mastocytes, basophils, eosinophils and neutrophils. There exists on the other hand a modulating activity of the response to stimulation by infectious agents or allergens of pro-inflammatory cells, mastocytes and basophils, which is disclosed in the state of the art.

However, no modulation of the inflammatory response of eosinophils and of neutrophils is disclosed to this day, i.e. no action before the release of proteases by these eosinophilic and neutrophilic cells is disclosed today. Indeed, all the studies conducted up to now target the action of the ovomucoid after secretion of proteases by pro-inflammatory cells, no study contemplating an action of the ovomucoid on neutrophils and eosinophils before release of the proteases.

The present invention relates to a particular use of the ovomucoid from eggs of the quail Corturnix corturnix japonica of strain B-Mina in an anti-inflammatory composition intended for humans and animals for modulating the cell response of neutrophils and eosinophils, i.e. which acts on the pro-inflammatory cells before the release of mediators of the inflammatory reaction like proteases. Within the scope of the present invention, it was actually observed quite surprisingly that the ovomucoid also acts as a modulator of the cell response of neutrophils and eosinophils before release of the proteases. This surprising action of the ovomucoid is particularly advantageous since it gives the possibility of directly acting on the two main pro-inflammatory cells involved during tissue inflammatory reactions for modulating the cell responses thereof and thus preventing the release of mediators such as proteases. This therefore allows minimization of the inflammatory reactions by acting before the release of proteases starting with neutrophils and eosinophils by the intracellular inhibition mechanism of the quail egg ovomucoid on neutrophils and eosinophils put forward within the scope of the present invention, as an addition to the anti-protease activity described in the state of the art and to the modulating response on mastocytes and basophils. This modulating activity is all the more surprising since it was not obvious to show a mechanism occurring upstream from a sought and expected phenomenon which should be caused in order to observe the release of proteases so as to be able to inhibit them. Further, the prior art is silent as regards the modulation of the cell response of eosinophils and neutrophils. Moreover, if the state of the art discloses the modulation of the cell response of mastocytes and of basophils, there is no indication that the mechanisms involved in the inhibition of the release of chemical mediators by mastocytes and basophils, by ovomucoids, would be identical for the inhibition of the release of chemical mediators by eosinophils and by neutrophils. If the state of the art discloses the inhibition of activation of basophils and the obtained results on mastocytes are inferred from the literature and relate to other anti-proteases, it surprisingly appeared that inhibition of the activation of eosinophils and neutrophils had taken place while these mechanisms are unknown to this day.

Advantageously, the anti-inflammatory composition according to the invention further comprises at least one vitamin selected from the group formed by vitamins A, B, C, D, E, F, K and P. These vitamins are known for their capability of reinforcing the immune system.

In a just as advantageous way, the anti-inflammatory composition according to the invention further comprises at least one mineral selected from the group formed by Al, Bi, Co, Cu, Fe, F, Li, Mg, Ni, P, K, Se, S and Zn. These minerals are also known for their capability of reinforcing the immune system.

Preferably, the anti-inflammatory composition according to the invention further comprises at least one amino acid selected from the group formed by Ile, Leu, Lys, Met, Phe, Thr, Trp, Val, Glu and Gln. These amino acids are also known for their capability of reinforcing the immune system.

Advantageously, the anti-inflammatory composition according to the invention further comprises at least one plant extract selected from the group formed by plant extracts of roots, stems, leaves, flowers and fruit.

Preferentially, the anti-inflammatory composition according to the invention further comprises a pharmaceutically acceptable excipient or an excipient compatible with food.

Preferably, the anti-inflammatory composition according to the invention is packaged in the form of tablets, globules, gelatin capsules, granules, powder, fluids, liquids, creams or sprays.

Advantageously, the anti-inflammatory composition according to the invention comprises Zn in a protected form, for example Zn bisglycinate (Zn is protected between two glycines). Indeed, it appeared that Zn in the protected form, in particular Zn bisglycinate, acts both on the immunity and chronic inflammation by inducing the synthesis of specific cytokines and by antagonism of oxidative stress as a fundamental element of the enzymes involved in the metabolism of oxygenated radicals.

Advantageously, the anti-inflammatory composition according to the invention is administered in the form of a food supplement.

Other embodiments of the anti-inflammatory composition are mentioned in the appended claims.

Other features, details and advantages of the invention will become apparent from the description given hereafter, as non-limiting and referring to the examples and to the appended figures.

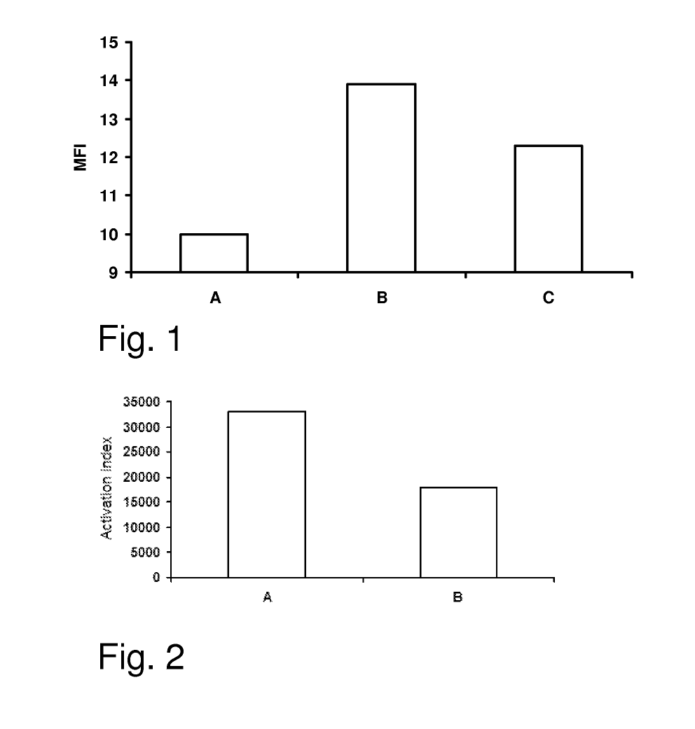

FIG. 1 illustrates the effect of the ovomucoid from eggs of the quail Corturnix corturnix japonica of strain B-Mina at a concentration of 10 μg/ml on the activation of eosinophils by the bacterial peptide fMLP.

FIG. 2 illustrates the effect of the ovomucoid from eggs of the quail Corturnix corturnix japonica of strain B-Mina at a concentration of 10 μg/ml on the activation of neutrophils by the bacterial peptide fMLP.

Exaggerated inflammatory reactions which may be pathogenic for humans are called hypersensitivity reactions. Since 2003, and on the recommendations of the Clinical Allergology and Immunology European Academy, a distinction has been made between immune hypersensitivities (antibodies depending on or involving cells of the immune system such as lymphocytes) and non-immune hypersensitivities not involving elements of the immune system and directly generating pro-inflammatory elements or having a direct toxic action.

In immuno-allergology, common allergens such as molds, cockroaches or mites show proteolytic activity, cysteine proteases for the allergens Der f1 and Der p1 and serine protease for allergens Der p3, 6, 9. These proteases are active via PAR (protease activated receptors) receptors and are involved in the recruitment of eosinophils and in bronchial hyperactivity in a mouse model of allergic inflammation.

The main blood cells having a fast pro-inflammatory action (of less than one hour) but being also involved in delayed mechanisms, are basophils, neutrophils and eosinophils.

Eosinophilia is essentially tissue eosinophilia and particularly at the digestive level. The main causes of hyper-eosinophillias are hypertopical eczema, allergic rhinitis and asthma, but many digestive pathologies are known today, in which a very significant recruitment of eosinophils is observed. The role of the eosinophil in immunomodulation is recognized today. This cell is rapidly present on the site of the inflammatory reaction and is essentially localized at the mucosas.

The eosinophil contains many cytotoxic mediators, such as eosinophil peroxydase (EPO), which are released by degranulation but the inhibitors of the degranulation of eosinophils are not very known. The eosinophils isolated from asthmatic patients contain a high activity of the chymotrypsin type which seems to involve these proteases in the degranulation mechanisms. Thus, nafamostat mesylate, a powerful inhibitor of serine-proteases, not only inhibits proteolytic activity but also increases the number of eosinophils in a BAL (broncho-alveolar lavage). Also, the SLPI (secretory leukocyte protease inhibitor) inhibits, in a conjunctival allergy model, development of conjunctivitis in guinea pigs.

The allergic inflammation is broken up into two main phases, i.e. an immediate phase and a belated phase. The deleterious effect of eosinophils is especially involved in the belated phase and when passing to chronicity. Intercellular interactions are multiple and a powerful pro-inflammatory pair is represented by the eosinophil-mastocyte pair.

The eosinophil is a blood cell which is found in tissues on the site of inflammatory reactions, spontaneously colonizing the mucosas and provided with a characteristic functional duality, participating in the anti-tumoral and parasite defence but also playing a major regulatory role on the immune system. This cell described for the first time by Paul Ehrlich (1854-1915) is characterized by the red brick color taken by its granules colored with eosin, because of the presence of strongly basic proteins.

The eosinophil has membrane receptors for IgGs (FcyRI and FcyRII), for IgAs (FcaRI) and for IgEs (FcεRI and FcεRII) as well as for various cytokines (IL2, IL3, IL5, IFNγ). Activation of eosinophils results in their degranulation with release of many mediators: mediators among which are proteins (MBP (major basic protein), ECP (eosinophil cationic protein) and EDN (eosinophil derived neurotoxin) and EPO (eosinophil peroxidase) enzymes, collagenases, β-glucuronidase, lysophosphatase, arylsulfatase and acid phosphatase, phospholipases, histaminases, catalases, esterases, hexosaminidases. Activation of eosinophils further leads to the secretion of oxygenated radicals of oxidative stress. They also release LTC4, PAF and derivatives from the cycloxygenase route. Further, they produce many cytokines and particularly immuno-regulatory cytokines (IFNγ, IL2, IL12, IL4, IL10, IL3), pro-inflammatory cytokines (IL1, IL6, TNFα), anti-inflammatory cytokins (TGFα and β) and chemokines (RANTES, eotaxin).

Activation of eosinophils may be mediated by the IgEs, IgGs or IgAs, the latter may even be in certain situations more activating than IgE and is often underestimated.

The neutrophil occupies a central position as an essential defence and pro-inflammatory cell. This cell is involved in oxidative stress since it may, like the eosinophil, release extremely active oxygenated derivatives and toxic for the tissues on the site of the inflammatory reaction.

The neutrophils may be activated by non-immune and immune routes and among the latter, via the route of Igs, IgGs and even IgEs. Indeed, neutrophils express the receptor with high affinity for IgE and are the first cells recruited after tissue damage, an infectious agent or allergens. Thus, bronchial allergenic challenge induces massive recruitment of neutrophils at the lung level in humans and animals.

Further, activated neutrophils synthesize IL8 during an IgE-dependent activation which induces an additional inflow of neutrophils. The neutrophil, like the eosinophil may release, after activation, proteolytic enzymes with deleterious effect on tissues.

It has therefore been demonstrated according to the present invention that the quail egg ovomucoid was capable of inhibiting, in addition to activation of the basophils induced by fMLP (N-formylmethionyl-leucyl-phenylalanine), a bacterial peptide having specific membrane receptors on human polynuclear cells, activation of eosinophils and neutrophils by fMLP.

### Example 1

### Inhibitory Effect of the Ovomucoid from Eggs of the Quail Corturnix corturnix japonica of Strain B-Mina on Eosinophils

A volume of 0.5 ml of blood is taken and diluted 3 times in a calcium buffer (127 mM NaCl, 5 mM KCl, 20 mM Trizma, 5 mM CaCl₂, 5 mM MgCl₂, 5 IU/ml heparin, pH 7.4) according to the following conditions:
negative control: 60 μl of calcium buffer are added to 60 μl of the previous solution (solution No.1);
positive control: 60 μl of the N-Formyl-Methionyl-Leucyl-Phenylalanine solution (fMLP 10-7M, Sigma, USA) are added to 60 μl of the previous solution (solution No.2);
test sample: 60 μl of the solution of the dilution of the ovomucoid from eggs of the quail Corturnix corturnix japonica of strain B-Mina at a concentration of 10 μg/ml are added to 60 μl of the previous solution (solution No. 3).

The thereby obtained solutions are pre-incubated for 30 mins at room temperature. Next, 60 μl of calcium buffer are added to solution No. 1 (negative control—block A in FIG. 1) while 60 μl of the N-Formyl-Methionyl-Leucyl-Phenylalanine solution (fMLP 10-7M, Sigma, USA) are added to the solutions No. 2 and No. 3 (blocks B and C in FIG. 1) before a second incubation for 30 mins at 37° C.

In each of the three samples, the leukocytes are specifically marked and incubated for 15 mins at room temperature and in darkness after adding a cocktail of marked antibodies (anti-IgE, anti-CD63, anti-CD11b, anti-CD49d). CD49d is a marker allowing selection of eosinophils, as well as negative selection of the neutrophils. The thereby prepared leukocyte suspensions are then lyzed (ammonium chloride buffer) before analysis (n=10) by flow cytometry (XL Coulter Beckman) allowing the cells to be characterized and sorted out, which are carried away by a flow in the beam of a laser, the re-emitted light giving the possibility of distinguishing the different types of cells. It is thus possible to distinguish whether the markers are expressed or not, i.e. if the eosinophils are either activated or not by measuring the fluorescence intensity (FI).

The results obtained by flow cytometry are illustrated in FIG. 1. The results indicate that the addition of ovomucoid to the solution comprising eosinophils and the bacterial peptide fMLP significantly (P<0.001) reduces the expression of the marker CD11b, which demonstrates the immunomodulating role of the ovomucoid on the cell response of the eosinophils. Indeed, the measured fluorescence intensity average for the samples containing the ovomucoids is significantly less than the measured fluorescence intensity average for the positive control. The decrease in the fluorescence intensity of the marker CD11b demonstrates inhibition of activation by fMLP. CD11b is an integrin very involved in communication with epithelial cells and has a major physiological role in the migration of eosinophils on the site of the inflammatory reaction. An inhibition of the expression of CD11b therefore corresponds both to an inhibition of cell activations and therefore potentially to the inhibition of the release of mediators but also to a decrease in the recruitment of these cells on the site of the inflammatory reaction.

### Example 2

### Inhibitory Effect of the Ovomucoid from Eggs of the Quail Corturnix corturnix japonica of strain B-Mina on Neutrophils

A volume of 0.5 ml of blood is taken and diluted 3 times in the calcium buffer (127 mM NaCl, 5 mM KCl, 20 mM Trizma, 5 mM CaCl₂, 5 mM MgCl₂, 5 UI/ml heparin, pH 7.4) according to the following conditions:
positive control: 60 μl of the solution of N-Formyl-Methionyl-Leucyl-Phenylalanine (fMLP 10-7M, Sigma, USA) are added to 60 μl of the preceding solution (solution No. 1);
test sample: 60 μl of the solution of the dilution of the ovomucoid from eggs of the quail Corturnix corturnix japonica of strain B-Mina at a concentration of 10 μg/ml are added to 60 μl of the previous solution (solution No. 2).

The thereby obtained solutions are pre-incubated for 30 mins at room temperature. Next, 60 μl of the N-Formyl-Methionyl-Leucyl-Phenylalanine solution (fMLP 10-7M, Sigma, USA) are added to the solutions No. 1 (blocks A in FIG. 2) and No. 2 (block B in FIG. 2) before a second incubation for 30 mins at 37° C.

In each of the three samples, the leukocytes are specifically and incubated for 15 mins at room temperature and in darkness after adding a cocktail of marked antibodies (anti-IgE, anti-CD63, anti-CD11b, anti-CD49d). Thus it was possible to detect the specific response of neutrophils by measuring the activation index of the marker CD11b. These results are shown in FIG. 2. These results show that the ovomucoid from eggs of the quail Corturnix corturnix japonica of strain B-Mina allow specific inhibition of the cell response of neutrophils by reducing their activation by the fMLP peptides.

### Potential Applications

Atopical dermatitis is an inflammatory disease of the skin which affects between 10 to 15% of children and the prevalence of which is steadily increasing. More than ⅔ of the patients begin their affection in the first year of life. As regards the natural history of the disease, the authors do not agree: some state that 90% of the atopical dermatitises are in remission before the age of 10 while others state that only 60% of these children will no longer suffer from atopy during adulthood. The truth is probably between both of these approaches, being aware that hospital studies are biased, small eczemas not arriving in hospital services. The poor prognosis criteria would be the beginning at an early age of severe signs, the association with a respiratory allergy of the asthma or allergic rhinitis type. Among the different immunological assumptions, disequilibrium of the Th1/Th2 balance to the benefit of cytokines of type 2, being at the origin of the development of an atopical dermatitis, is supported by studies showing the existence in infants of a high serum level of IL4 for several months before the triggering of the atopical dermatitis. IL5, a cytokine in majority involved in tissue recruitment of eosinophils, will also be intimately related to the development of atopical dermatitis.

The treatment of this pathology is based on the associated uses of topical drugs, of a suitable diet and of chemotherapy from corticoids to immunosuppressants.

The eosinophil is involved in many pathologies, such as gastritis, esophagitis and eosinophilic proctocolitis and other intestinal inflammatory diseases such as ulcerative colitis and Crohn's disease.

Mention should also be made of the Churg-Strauss syndrome, of eosinophilic pneumonia and of course parasite infestations. However, the most frequent causes of eosinophilia, in the western world, are the allergic inflammatory diseases such as atopical dermatitis, allergic rhinitis and asthma. In these pathologies, the eosinophil does not act alone but many cell cooperations are demonstrated today such as the eosinophil-mastocyte cooperation.

The ovomucoid further has an immunomodulating potential, and effects on the production of T-cell dependent cytokines have been described. The deleterious effect of proteases from the activation of the neutrophils on the lung mucosa has led to the search for specific therapeutic molecules. It was also demonstrated that the inhibition of the proteases of the neutrophil induced better efficiency of phagocytosis by increasing the oxgenative burst. In this way, an application of ovomucoid in the treatment of atopical dermatitis and similar affections gives the possibility of benefiting from anti-protease intracellular action of the ovomucoid of quail eggs, which is added to its natural extracellular activity. Thus, the quail egg ovomucoid shows an inhibitory effect on the activation of human polynuclear cells (eosinophils, basophils, neutrophils) and is therefore an excellent anti-inflammatory candidate not only for atopical dermatitis.

It is quite understood that the present invention is by no means limited to the embodiment described above and that many modifications may be made thereto without departing from the scope of the appended claims.

1. An anti-inflammatory composition intended for humans and animals for modulating a cell response of neutrophils and eosinophils, the anti-inflammatory composition comprising: an extract from eggs of a quail Corturnix corturnix japonica of a strain B-Mina comprising an ovomucoid and a quail egg ovo-inhibitor.

2. The anti-inflammatory composition according to claim 1, further comprising: at least one vitamin selected from a group consisting of A, B, C, D, E, F, K and P.

3. The anti-inflammatory composition according to claim 1, further comprising: at least one mineral selected from a group consisting of Al, Bi, Co, Cu, Fe, F, Li, Mg, Ni, P, K, Se, S and Zn.

4. The anti-inflammatory composition according to claim 1, further comprising: at least one amino acid taken from a group consisting of Ile, Leu, Lys, Met, Phe, Thr, Trp, Val, Glu and Gln.

5. The anti-inflammatory composition according to claim 1, further comprising: at least one plant extract selected from a group formed by plant extracts from roots, stems, leaves, flowers and fruit.

6. The anti-inflammatory composition according to claim 1, further comprising: a pharmaceutically acceptable or food-compatible excipient.

7. The anti-inflammatory composition according to claim 1, wherein the anti-inflammatory composition is packaged as one of tablets, globules, gelatin capsules, granules, powders, fluids, liquids, creams and sprays.

8. The anti-inflammatory composition according to claim 3, wherein Zn is protected Zn.

9. The anti-inflammatory composition according to claim 1, wherein the anti-inflammatory composition is in a form of a food supplement.

10. The anti-inflammatory composition according to claim 2, further comprising: at least one amino acid taken from a group consisting of Ile, Leu, Lys, Met, Phe, Thr, Trp, Val, Glu and Gln.

11. The anti-inflammatory composition according to claim 2, further comprising: at least one plant extract selected from a group formed by plant extracts from roots, stems, leaves, flowers and fruit.

12. The anti-inflammatory composition according to claim 2, further comprising: a pharmaceutically acceptable or food-compatible excipient.

13. The anti-inflammatory composition according to claim 2, wherein the anti-inflammatory composition is packaged as one of tablets, globules, gelatin capsules, granules, powders, fluids, liquids, creams and sprays.

14. The anti-inflammatory composition according to claim 2, wherein the anti-inflammatory composition is in a form of a food supplement.

15. The anti-inflammatory composition according to claim 2, further comprising: at least one mineral selected from a group consisting of Al, Bi, Co, Cu, Fe, F, Li, Mg, Ni, P, K, Se, S and Zn.

16. The anti-inflammatory composition according to claim 15, further comprising: at least one amino acid taken from a group consisting of Be, Leu, Lys, Met, Phe, Thr, Trp, Val, Glu and Gln.

17. The anti-inflammatory composition according to claim 16, further comprising: at least one plant extract selected from a group formed by plant extracts from roots, stems, leaves, flowers and fruit.

18. The anti-inflammatory composition according to claim 17, further comprising: a pharmaceutically acceptable or food-compatible excipient.

19. The anti-inflammatory composition according to claim 18, wherein the anti-inflammatory composition is packaged as one of tablets, globules, gelatin capsules, granules, powders, fluids, liquids, creams and sprays.

20. The anti-inflammatory composition according to claim 19, wherein the anti-inflammatory composition is in a form of a food supplement.
